(19) United States
(12) Patent Application Publication
(10) Pub. No.: US 2015/0238730 A1
(43) Pub. Date: Aug. 27, 2015

(54) ULTRASOUND VISIBLE CATHETER

(71) Applicant: The George Washington University, Washington, DC (US)

(72) Inventors: Adam Corman, McLean, VA (US); Neal K. Sikka, Vienna, VA (US); Nathan J. Castro, Washington, DC (US); Benjamin Holmes, Washington, DC (US); Hawaa Almansouri, Abu Dhabi (AE)

(21) Appl. No.: 14/633,960

(22) Filed: Feb. 27, 2015

Related U.S. Application Data
(63) Continuation-in-part of application No. PCT/US2014/018931, filed on Feb. 27, 2014.
(60) Provisional application No. 61/945,611, filed on Feb. 27, 2014.

(51) Int. Cl. A61M 25/01; A61B 8/08

(52) U.S. Cl. CPC A61M 25/0108; A61B 8/0841

(57) ABSTRACT

A catheter can be used in arterial and/or venous placement, in conjunction with ultrasound positioning. The catheter itself is made of the same materials, and is sized using the standard gauges, as current existing catheters. However, the catheter has a microtexture on the outer surface of the catheter. This microtexture efficiently and effectively reflects propagated ultrasound waves in situ, to provide a brighter and clearer image of the catheter when being placed in a patient's vessel, with ultrasound imaging equipment.

### RELATED APPLICATIONS

This application is a continuation-in-part of PCT/US2014/018931, filed Feb. 27, 2014, which claims benefit of U.S. Provisional Application No. 61/770,052, filed Feb. 27, 2013. This application also claims the benefit of U.S. Provisional Application No. 61/945,611, filed Feb. 27, 2014. The entire contents of each of those applications are incorporated herein by reference.

### BACKGROUND OF THE INVENTION

1. Field of the Invention

The present invention relates to catheters and the placement of catheters in a patient. More particularly, the present invention relates to a catheter that is visible to ultrasound to assist placement of the catheter in a patient.

2. Background of the Related Art

Catheters are small tubes that can be inserted into the body for medical purposes, such as to introduce or remove a gas or fluid for the treatment of an illness or disease or to perform a surgical procedure. The process of inserting a catheter into a body cavity, duct or vessel is called catheterization. Correct placement is essential for the catheter to operate properly.

Catheter introduction sets are generally known. For instance, U.S. Pat. No. 4,417,886 discloses a catheter introduction set having a needle, catheter, wire guide and wire guide feed device in which when the needle is positioned into a lumen of a blood vessel, a wire guide is first inserted into the vessel and the catheter is fed over the wire guide from the use of a radially extended handle into the lumen. This arrangement is similarly disclosed in U.S. Pat. No. 4,772,264 with the addition of a retaining finger for stabilization of the catheter on the skin.

Catheters (including central and peripheral catheters) are not designed to be visualized under ultrasound. Properties of current catheter materials are not acceptable for ultrasound detection. A catheter can be placed into a vessel with an introducer needle with or without a guide wire. The guide wire and the introducer needle can be visualized under ultrasound, but the catheter itself cannot be visualized. Because the catheter cannot be visualized, sometimes catheters are placed incorrectly. Examples of incorrect catheter placement include: in an artery instead of a vein, in a vein instead of an artery, and in neither a vein nor an artery. Arteries flow next to veins and can be easily catheterized by accident when aiming for a vein. Accidental placement of a catheter in an artery instead of a vein can lead to destruction of tissues receiving blood from that artery due to application of medications that damage arteries and are intended only for use in veins.

Ultrasound guided catheters are more often in deeper vessels. This is because vessels closer to the surface are more easily visualized leading to greater success with the conventional technique. With the deep and small nature of the vessels used in the ultrasound guided IV catheter technique it can take more time and attention to place these catheters. More of the catheter is in the soft tissue between the skin surface and the deep vessel, which means that the deeper vessel has less of the catheter within its lumen.

Microtextures have been investigated to enhance or inhibit biological interactions of catheters (e.g. antithrombic possessing enhanced drug eluding capabilities [2] and cell ingrowth inhibition [3]), but no one has employed texturing on vascular catheters to make them more reflective/visible to ultrasound. The identification of texture in medical imaging can be used to differentiate between different tissues and materials in the body [4-6]. Furthermore, microtextures and micro-featured materials have been widely used in other fields to absorb and reduce acoustic and fluid waves or for the use of acoustic texture recognition for advanced navigation [7, 8].

### SUMMARY OF THE INVENTION

Accordingly, it is an object of the invention to provide a catheter that can be visualized by ultrasound. It is a further object of the invention to provide a catheter with an outer texture that reflects ultrasound so that a medical professional can visually see the catheter as it is being placed in the patient to verify proper placement. Another object of the invention is to provide a catheter (including central and peripheral) that is easily visualized by ultrasound, to enable correct placement to be determined and recorded by ultrasound visualization.

In accordance with these and other objects of the invention, a catheter is provided for use in arterial and/or venous placement, in conjunction with ultrasound positioning. The catheter itself is made of the same materials, and is sized using the standard gauges, as current existing catheters [1]. However, the catheter has a microtexturing on the outer surface of the catheter (added either during fabrication or post-fabrication). This microtexture efficiently and effectively reflects propagated ultrasound waves in situ, to provide a brighter and clearer image of the catheter when being placed in a patient's vessel, with ultrasound imaging equipment.

These and other objects of the invention, as well as many of the intended advantages thereof, will become more readily apparent when reference is made to the following description, taken in conjunction with the accompanying drawings.

### BRIEF DESCRIPTION OF THE FIGURES

FIG. 1 is a side view of a catheter in accordance with an embodiment of the invention;

FIG. 2 is an enlarged perspective view of the catheter tube of FIG. 1 having a microtexture formed by hexagonal projections;

FIG. 2A is an enlarged view of the microtexture for the catheter of FIGS. 1 and 2;

FIG. 2B is a cross-sectional side view of the catheter and microtexture;

FIG. 3 is a perspective view of another embodiment of the invention in which the microtexture is formed by round dimples;

FIG. 3A is an enlarged view of the microtexture for the catheter of FIG. 3;

FIG. 3B is a cross-sectional side view of the catheter and microtexture of FIGS. 3, 3A;

FIG. 4 is a perspective view of another embodiment of the invention in which the microtexture is formed by a recessed V-shape;

FIG. 4A is an enlarged view of the microtexture for the catheter of FIG. 4;

FIG. 4B is a cross-sectional side view of the catheter and microtexture of FIGS. 4, 4A;

FIG. 5 is a perspective view of another embodiment of the invention in which the microtexture is formed by an inverted pyramid shape;

FIG. 5A is an enlarged view of the microtexture for the catheter of FIG. 5;

FIG. 5B is a cross-sectional side view of the catheter and microtexture of FIGS, 5, 5A;

FIG. 6 is a perspective view of another embodiment of the invention in which the microtexture is formed by a recessed diamond shape;

FIG. 6A is an enlarged view of the microtexture for the catheter of FIG. 6; and

FIG. 6B is a cross-sectional side view of the catheter and microtexture of FIGS. 6, 6A.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in similar manner to accomplish a similar purpose. Several preferred embodiments of the invention are described for illustrative purposes, it being understood that the invention may be embodied in other forms not specifically shown in the drawings.

Reflective Patterns

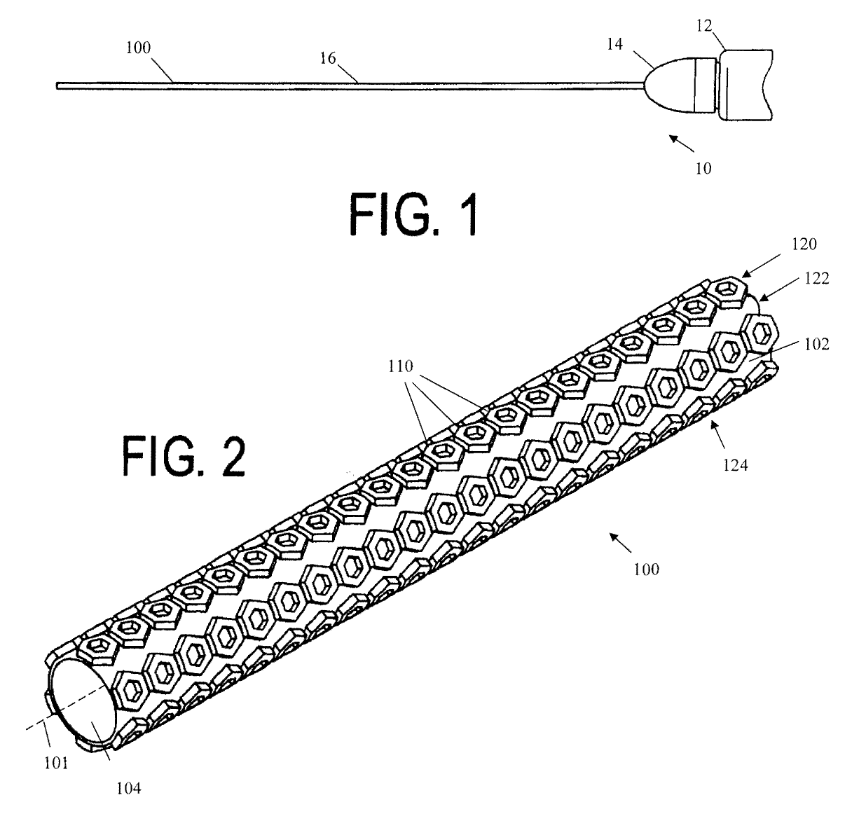

Referring to the drawings, FIG. 1 shows the catheter/needle assembly 10 of the present invention. The assembly 10 includes a round tubular housing 12, hub 14, needle 16 and catheter 100. The housing 12 has a first end that is open and can mate with a medication container or drain. The needle 16 can extend through openings in the hub 14 and housing 12. The catheter 100 extends over the outside of the needle 16 and terminates at and couples with the hub 14. It should be noted, however, that the catheter 100 of the present invention can be provided with any suitable assembly 10, such as the housing 12, hub 14 and needle 16 of FIG. 1. In addition, the catheter 100 can be provided by itself.

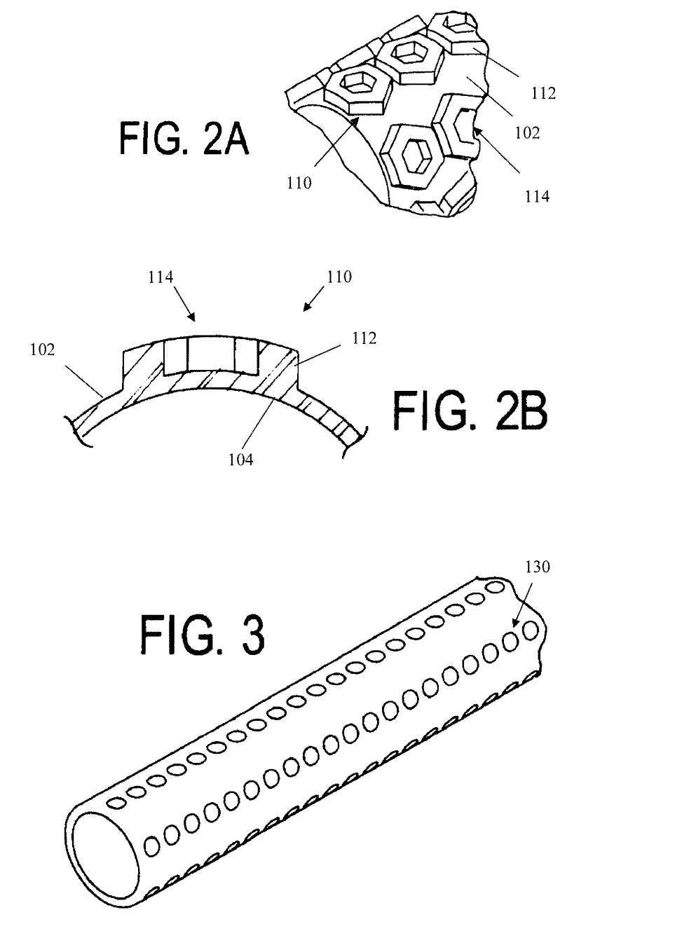

Turning to FIG. 2, the catheter 100 is shown in greater detail. The catheter 100 has a tubular and elongated catheter body with a central longitudinal axis 101. It has an outer surface 102 on the outside of the tube and an inner surface 104 opposite the outer surface 102 at the inside of the tube. A microtexture or pattern is formed on the outer surface 102. As shown, the texture can be one or more hexagonal shaped reflective members 110 that form a repeating pattern. Here, the reflective members 110 are aligned in a plurality of columns 120 that extend along the entire length of the outer surface 102 of the catheter 100, parallel to the longitudinal axis 101. Several columns 120 of reflective members 110 are positioned about the outer surface 102, and six columns 120 are shown in the illustrative embodiment of FIG. 2. The columns 120 can be separated from each other by a gap 122 so that the reflective members 110 do not touch each other. The reflective members 110 are sized in the micro-range, on the order of up to about several hundred microns; though other suitable sizes can be utilized depending on the size of the substrate (catheter) and the desired application.

As best shown in FIG. 2A, the reflective members 110 can have a hexagonal shape. As further shown in FIG. 2B, the reflective members 110 can project outward from the outer surface 102 of the catheter 100 to form a wall 112. The reflective members 110 can be solid or can have a central opening 114 that can have the same shape as the outer periphery of the reflective members 110 (here a hexagon). The reflective members 110 are also aligned in rows 124 that extend transversely across the catheter 100 and perpendicular to the longitudinal axis 101. The reflecting members 110 in neighboring rows 124 can be aligned with each other, or alternating columns 120 can be shifted so that the reflecting members 110 in neighboring rows 124 are offset from one another and the reflecting members 110 in alternating columns 120 are aligned. Still yet, the reflecting members 110 need not be positioned in columns 120 or rows 124 and for instance the reflecting members 110 can be positioned randomly about the outer surface 102. The wall 112 is roughly 50 microns tall, but may be several microns shorter.

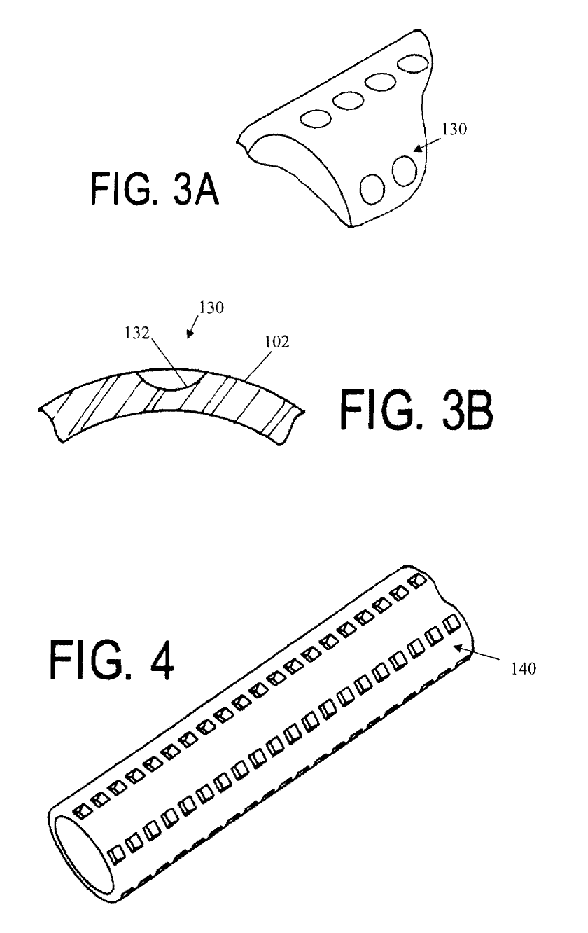

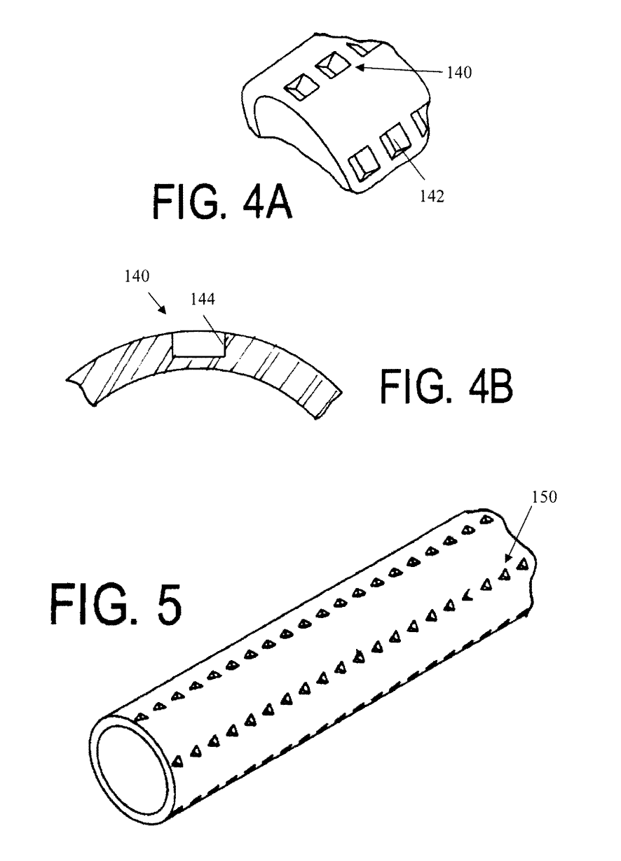

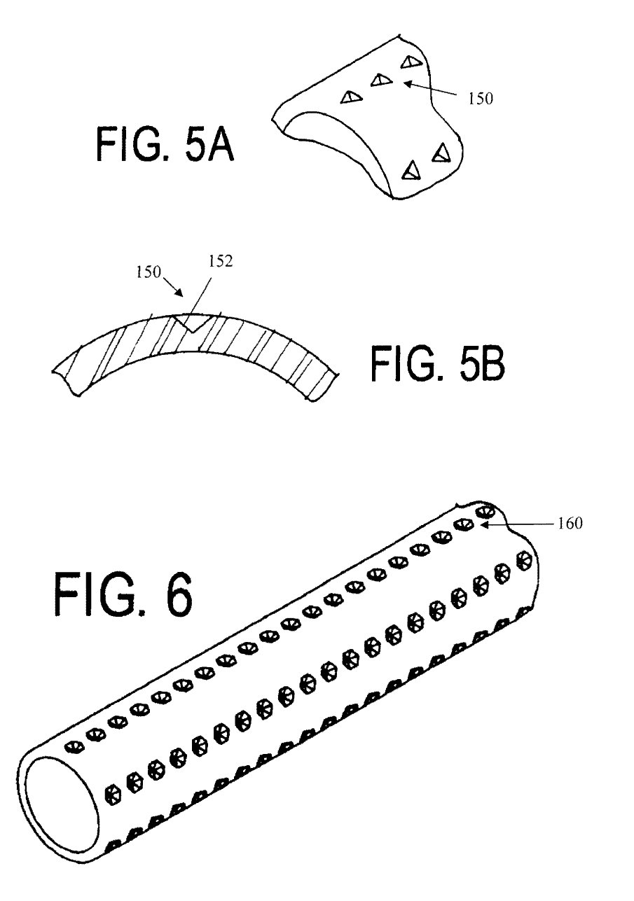

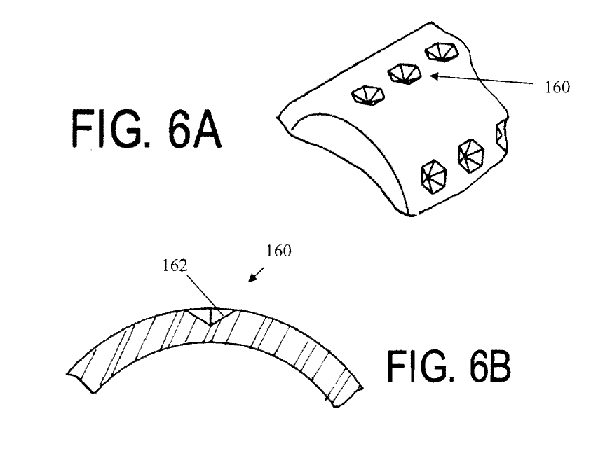

Referring to FIGS. 3-6, several alternative embodiments of the invention are shown, each having a respective microtextured pattern that is highly visible to ultrasound. The patterns include reflective members 130-160 with different shapes, namely round dimples members 130 (FIGS. 3, 3A, 3B), recessed V-shaped members 140 (FIGS. 4, 4A, 4B), inverted pyramid members 150 (FIGS. 5, 5A, 5B), and inverted diamond-shaped members 160 (FIGS. 6, 6A, 6B). All of those patterns of are based on a reflective lens structure (i.e., the reflective members 130-160 are each configured to operate as a reflective lens). Thus, the reflective members 130-160 are all concave, multi-sided shapes that are recessed in the outer surface 102 of the catheter 100. The reflective members 130-160 are designed to capture, amplify and reflect incoming acoustic waves of an ultrasound signal. The reflective members 130-160 can be positioned about the outer surface 102 as described with respect to FIGS. 2, 2A, 2B. In addition, because the reflective members are recessed, they do no interfere with movement of the catheter within the lumen of a blood vessel and do not otherwise increase the outer diameter or decrease the inner diameter of the catheter 100.

The depth of each reflective member 130-160 (concave multisided shape) can run from 250 microns to 50 microns, and the width may go from 500 microns to 200 microns. Additionally, depending on the catheter size (typically 18 to 22 (1.27 to 0.711 mm OD) gauge for peripheral lines, and 14 or 15 gauge (2.108 to 1.829 mm OD) for central lines) a given catheter 100 could be covered with hundreds of textured reflective members 110, 130, 140, 150, 160. For instance, a typical 18 gauge catheter 100 can have 10-20 columns 120 of reflective members. The recesses should be sufficiently shallow as to not compromise the reliability of the catheter 100 to carry fluids.

Referring to FIGS. 3, 3A, 3B, the reflective members 130 are shown having a circular, semi-spherical shape. The reflective members 130 are formed as recesses in the outer surface 102 of the catheter 100 to form a curved reflective face 132. The reflective members 130 can have a radius of about 0.50 mm, a depth of 0.25 mm and a diameter of 0.86 mm at the outer surface 102. Turning to FIGS. 4, 4A, 4B, the reflective members 140 are recesses in the outer surface 102 of the catheter 100. The reflective members 140 have a corner shape formed by a 90° angle at two joining two straight reflective faces 142 lying in intersecting planes, and side walls 144 that are perpendicular to the outer surface 102. The depth is about 0.35 mm and the length at the outer surface 102 is about 0.71 mm. Referring to FIGS. 5, 5A, 5B, the reflective members 150 are shown having a cubic corner shape formed by three straight reflective faces 152 joining at right angles, as in the corner of a cube or an inverted pyramid. The reflective members 150 are formed as recesses in the outer surface 102 of the catheter 100. The reflective members 150 can have a maximum width of about 0.55 mm, a depth of 0.24 mm and a length of 0.56 mm at the outer surface 102 of the catheter 100. In FIGS. 6, 6A, 6B, the reflective members 160 have a diamond design, consisting of a hexagon with joining triangular straight reflective faces 162 at each face, descending to form a single point. The reflective members 160 are formed as recesses in the outer surface 102 of the catheter 100. The reflective members 160 can have a width of from 0.72-0.84 mm and a depth of about 0.25 mm.

Thus, the reflective members 130, 140, 150 and 160 are configured so that the respective reflective faces 132, 142, 152 and 162 are positioned to reflect impinging ultrasound waves which are then received by the detector of an ultrasound device, and displayed as an image.

Operation

Referring back to FIG. 1, the operation of the assembly 10 is described. Catheters (including central and peripheral) that are easily visualized by ultrasound, enable correct placement to be determined and recorded by ultrasound visualization. As an example, one could confirm by ultrasound that a catheter intended to be placed in a vein is in fact located in a vein and not in an artery or outside any blood vessel.

The user places the needle 16 into the patient, such as the vein or artery lumen. The needle 16 is aligned along the skin and then inserted into the lumen of the vessel. Once the needle 16 is properly placed, the user can optionally advance a guide wire (if provided) into the patient. The user can hold an ultrasound probe to assist placement of the guide wire and catheter 100. The guide wire enters the central opening in the needle 16 and continues through the needle 16 until it emerges from the distal tip of the needle 16 and enters the patient. At this point, the soft plastic catheter 100 is slid over the thin guide wire into the vessel lumen. Once the guide wire has entered the user's vein (which is roughly one to two and a half inches depending on catheter length), the user pushes the hub 14 (or finger tabs on the catheter 100, if used) forward to force the catheter 100 off the needle 16 and into the patient. The catheter 100 can be pushed into the patient further than the needle 16 and/or the guide wire since it is more flexible and won't puncture the side of the vein or vessel. The user can confirm the proper guidance and placement of the catheter 100 by visualizing it with ultrasound, then remove the needle 16 and guide wire from the patient, leaving the catheter 100 and hub 14 properly placed in the patient. The user can then connect a syringe, intra venous (IV) device or other medical instrument at the rear end of the hub 14.

### Conclusion

The catheter 100 is a single-piece member that is constructed only of materials that are standard for catheters, such as plastics, urethanes or rubbers. A typical material for catheters is Tecoflex EG-80A polyurethane. Accordingly, no other materials or metals are used so that the catheter 100 substantially retains its flexibility, reliability and functionality without added risk of infection. However, those materials cannot be detected by ultrasound because the sound velocities and impedance values of those materials are very low (0.959-2.06 km/s and 1.41-2.00 MRayl). The reflective members 110, 130-160 enable the catheter 100 to be visualized by ultrasound. The reflective members 110, 130-160 act as an interference pattern and/or reflector, so ultrasound acoustic waves, instead of traveling through the catheter material, are either slowed down or reflected, registering a signal on the ultrasound receiver and allowing the device to display an image of the catheter.

Thus, the catheter (which can be used for instance in arterial and/or venous placement) can be positioned using ultrasound. The catheter itself is made of the same materials, and is sized using the standard gauges, as current existing catheters [1]. However, the catheter has a microtexturing on the outer surface of the catheter. The reflective elements 110, 130-160 can be formed integrally with the catheter 100 (such as by molding or stamping integrated with the extrusion process) no that the catheter 100 is a single piece member. Or the reflective elements 110, 130-160 can be added during post-fabrication. This may be done either with a heated metal stamp or a roller or an extrusion dye which adds the texture to the catheter tubing as it is being extruded or soon after. The patterns may also be added using material subtraction techniques such as laser milling or photolithography. We can also employ chemical and vapor deposition to add the patterns, or through other additive techniques. This microtexture efficiently and effectively reflects propagated ultrasound waves in situ, to provide a brighter and clearer image of the catheter when being placed in a patient's vessel, with ultrasound imaging equipment.

As shown and described, the reflective members 110, 130-160 are positioned on the outer surface 102 of the catheter so that they do not obstruct the flow of gas or fluid inside the catheter 100. However, the reflective members 110, 130-160 can instead (or also) be provided at the inner surface 104 of the catheter 100. In addition, while the reflective members 110, 130-160 are collectively reflective of ultrasound, each reflective member 110, 130-160 alone need not be substantially reflective of ultrasound. Still further, the reflective pattern can be provided along the entire length of the catheter 100 or only at certain portions of the catheter 100 such as for several inches at the distal end of the catheter 100. And, while the patterns are separately shown in each of FIGS. 2-6, the patterns can be mixed together on a single catheter 100. And a single larger reflective member can be provided instead of a plurality of small reflective members.

It is further noted that the invention is described and shown as being used for a catheter 100. However, it will be appreciated that one or more the reflective members can be provided on other medical devices and non-medical devices. In addition, while the reflective members are configured (sized, shaped and form a pattern) to be visible to ultrasound, the reflective members can be configured to be visible to other frequencies and technologies, such as those used for X-ray or X-ray computed tomography (catscan).

The present invention can be utilized with any suitable ultrasound frequency, including within the typical ultrasound diagnostic frequency range of 2-20 MHz. The catheter 100 will be reliably visual by ultrasound if about 10% or more of the signal is reflected by the microstructures. However, there is no standard value for what amount of ultrasound should be reflected to produce a usable diagnostic image. A skilled eye may be able to identify shapes and structures on an image at lower reflection percentages.

As described in one or more of the related applications noted above, the entire contents (including the figures and written description) of which are herein incorporated by references, the catheter 100 can be constructed of materials that can be easily visualized under ultrasound. For instance metals, ceramics, or compounds (like barium sulfate or titanium), can be integrated within the structure of the catheter material allowing for direct visualization of catheter placement with the ultrasound. In yet another embodiment, the outer surface of the catheter 100 can be coated with metallic material such as Titanium, the catheter. This makes the catheter 100 detectable by ultrasound, because the high impedance surface will reflect some of the sound. In addition, the catheter 100 (or at least a portion of it) can not only be constructed of and/or coated with ultrasound-visible materials, but it can also have one or more reflective members or pattern. At least a portion of the catheter body and/or the reflective members can be made of or coated with the ultrasound-visible materials.

The following documents are incorporated herein by reference: (1) Systems, B.A., Central Venous Catheters. Bard Product Catalog 2014; (2) Lewandowski, J. J., et al., Development of an implantable drug delivery catheter. ASAIO Trans, 1991. 37(3): p. M295-7; (3) Walboomers, X. F. and J. A. Jansen, Effect of microtextured surfaces on the performance of percutaneous devices. J Biomed Mater Res A, 2005. 74(3): p. 381-7; (4) Rolland, Y., et al., Analysis of texture in medical imaging. Review of the literature. Ann Radiol (Paris), 1995. 38(6): p. 315-47; (5) Kern, R., et al., Characterization of carotid artery plaques using real-time compound B-mode ultrasound. Stroke, 2004. 35(4): p. 870-875; (6) Pingitore, A., et al., Stress-induced changes in subendocardial tissue texture in hypertrophic cardiomyopathy: An echocardiographic videodensitometric study. International Journal of Cardiovascular Imaging, 2001. 17(4): p. 245-252; (7) Chapagain, K. R. and A. Ronnekleiv, Grooved backing structure for CMUTs. IEEE Trans Ultrason Ferroelectr Freq Control, 2013. 60(11): p. 2440-52; (8) Politis, Z. and P. J. P. Smith, Classification of textured surfaces for robot navigation using continuous transmission frequency-modulated sonar signatures. International Journal of Robotics Research, 2001. 20(2): p. 107-128.

The foregoing description and drawings should be considered as illustrative only of the principles of the invention. The invention may be configured in a variety of shapes and sizes and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A catheter comprising:
an outer surface; and
at least one reflective member provided on the outer surface, said at least one reflective member configured to reflect an ultrasound signal.

2. The catheter of claim 1, wherein said at least one reflective member extends outwardly from said outer surface.

3. The catheter of claim 1, wherein said at least one reflective member is a recess in said outer surface.

4. The catheter of claim 1, further comprising a plurality of reflective members provided on the outer surface.

5. The catheter of claim 4, wherein said plurality of reflective members form a pattern on the outer surface that reflects an ultrasound signal.

6. The catheter of claim 1, wherein the catheter is made entirely of polyurethane.

7. A catheter comprising:
a catheter body having an outer surface; and
a plurality of reflective members provided on the outer surface of said catheter body, the plurality of reflective members forming a pattern that reflects an ultrasound signal.

8. The catheter of claim 7, wherein said reflective member extends outwardly from said outer surface.

9. The catheter of claim 7, wherein said reflective member is a recess in said outer surface.

10. The catheter of claim 7, wherein the catheter is made entirely of polyurethane.

11. The catheter of claim 7, wherein the catheter body is tubular.
